(19) United States
(12) Patent Application Publication
(10) Pub. No.: US 2025/0169753 A1
(43) Pub. Date: May 29, 2025

(54) Weight, Force, and Acceleration Measurements

(71) Applicants: William Tran, Chicago, IL (US); Bo Tran, Chicago, IL (US)

(72) Inventors: William Tran, Chicago, IL (US); Bo Tran, Chicago, IL (US)

(21) Appl. No.: 18/593,142

(22) Filed: Mar. 1, 2024

Related U.S. Application Data
(60) Provisional application No. 63/489,028, filed on Mar. 8, 2023.

(51) Int. Cl. A61B 5/00; A61B 3/113; A61B 5/11

(52) U.S. Cl. CPC A61B 5/486; A61B 3/113; A61B 5/1126; A61B 5/4848; A61B 2562/0219

(57) ABSTRACT

Methods of improving performance or attaining goals or tasks are disclosed, utilizing instantaneous data on mass, acceleration, and force. Applications of accelerometers are discussed. Applications can be useful for individuals or athletes, machinery or equipment, and animals.

### BACKGROUND OF THE INVENTION

Problem Solved: This invention provides individuals and/or athletes a method to track and enhance their exercise, goal, and routine. Measurements of weight, force and acceleration associated with the individual exercise or task are tabulated wirelessly informing the individual of their present and historical statistics, and what they need to perform or weight to attain certain goals. Data is provided instantaneously using a smart device.

Other applications of the present inventions are described below.

### DETAILED DESCRIPTION OF THE INVENTION

This invention relates generally to methods of providing individuals including athletes with metrics to help them improve their exercise routine including metrics that can be applied to attain specific goals.

Present exercising equipment, the likes of treadmills, bikes, stair climbers, ellipticals, and rowing machines, records athletes performances when they are utilizing the equipment. Data provided includes: calories, speed, and watts or resistance. These present exercise equipment also provides virtual coaching, pushing the individuals to perform better, and perhaps comparing the individual performance with other competitor(s) virtually.

There is still a need to combine the individuals or objects' weight, motion, and force to improve the individuals' skills. And there is a need to obtain this information instantaneously. The present invention provides a novel solution that provides instantaneous data of force or energy, and acceleration to move a weighted object or an individual's weight. Adjustments to the parameters can provide an individual insight to what is required to accomplish a certain task or attain a goal.

The present invention utilizes an accelerometer, imputed weight of the individual or object, and determines the force, inertia, or energy required to perform or meet the task or individual's goal or objective. Reversely, knowing the force and acceleration, an individual can gain knowledge of the weight he or she can bear to attain his or her goals.

Examples: If a person wanted to lift weights based on force or acceleration required, the present invention can provide information to the individual or athlete on how fast to lift the weight and the force the athlete would need to lift a 100 pound weight, versus lifting a 400 lb weight that will take more strenuous effort and time to perform. A weight sensing device can be attached to the weighted object, or the weight can be imputed manually. Another example, if a basketball player wants to track how much energy or force is needed (from his leg muscles) based on his weight input and acceleration, the present invention can provide this information. The basketball player can track and determine how high he or she can jump based on the instantaneous data collected by the present invention. Or, another example, what is the energy and acceleration needed for an individual, with a specific weight, to dunk a basketball in a basketball rim 10 feet high. Utilizing the individual's weight, which may increase or decrease, the present invention can gauge the energy and acceleration requirements to meet the individual's goal or objective. The individual will know how much weight he or she needs to gain or lose to achieve his or her goal. Or how much body force is needed to dunk a basketball. And the data can be provided instantaneously.

The present invention utilizes an integrated accelerometer and weight input (using a weight sensor or inputted manually) to calculate the energy or force. The accelerometer can be a small device or chip that can be inserted into an individual or animal's body. This metric can be used to track and gauge the individual's performance. Data is provided instantaneously to a smart device. The smart device can be a cellular phone.

An illustrative example where the present invention comprises a “velcro” type strap that is attached to an individual, animal, equipment, or machine. This strap or attachment contains an accelerometer. The strap can also have other elements consisting of a printed circuit board and a rechargeable lithium battery. The printed circuit board performed the analysis of weight, force, and acceleration.

The principle of the present invention is based on Newton's second law of motion: force is equal to mass multiplied by acceleration. If two of the three variables are known, the third variable can be calculated. Variations of Newton's law can be applied. Softwares is installed or utilized in the present invention to perform the calculations. The present invention's novelty is inputting the individual or object's weight and having acceleration data to calculate the force or energy required to complete a task. Weight can be monitored through using a sensor, or inputted manually. The data or calculations are released instantaneously. What if scenarios can be derived by changing one or more of the variables (force, mass, and/or acceleration). The novelty of this invention allows the individual with instantaneous data on what he or she needs to do to reach certain achievements or to attain specific goals.

The present invention can be applied to machinery and equipment—not only to individuals. For example, if information is needed to move objects of different weights, data can be imputed to determine the force and acceleration needed to accomplish the immediate task. What if scenarios can be performed ie., variables of force, acceleration, and/or weight can be changed to determine the output. The information can be provided instantaneously.

The present invention can be applied to animals. For example, if information is needed to evaluate an animal's potential to move at a certain speed based on variable weights of the animal, data can be imputed to determine the force and acceleration needed to accomplish the task or goal. For example, the present invention can be applied to race horses to improve its performance. An accelerometer can be inserted into the horse's body or leg. What if scenarios can be performed ie., variables of force, acceleration, and/or weight can be changed to determine the output. The information can be provided instantaneously.

1-12. (canceled)

13. A method to measure and calculate an individual animal, human, equipment, or machine's mass, energy or force, and/or acceleration to accomplish a feat or goal wherein the measurements of weight, force and acceleration are tabulated wirelessly, and wherein data is calculated, provided, or recorded instantaneously using a smart device.

14. The method of claim 13 wherein the weight is imputed to calculate force or energy and acceleration needed to accomplish a task or goal.

15. The method of claim 13 wherein the weight, force, and acceleration are calculated simultaneously and provided instantaneously.

16. The method of claim 13 that utilizes an accelerometer, weight input and electronic circuitry with data storage capability to perform instantaneous measurements of force or energy, acceleration, and weight to accomplish a certain task or to attain a goal.

17. The method of claim 16 wherein the accelerometer can be inserted or transplanted into a machine, a human or an animal's body.

18. The method of claim 17 wherein the accelerometer is used to determine behavior and provide a proxy for movement-based energy expenditure.

19. The method of claim 17 wherein the accelerometer replaces or enhances the vestibular organ in the inner ear of an animal or human.

20. The method of claim 17 wherein the accelerometer provides the human or animal brain with information on the motion and orientation of the body.

21. The method of claim 17 wherein the accelerometer is used to study therapeutics designed to treat degenerative joint disease (DJD) associated with pain in an animal or human.

22. The method of claim 17 wherein the accelerometer is used to measure the pupil activity of the human or animal throughout the day.
